(19) United States
(12) Patent Application Publication
(10) Pub. No.: US 2023/0073725 A1
(43) Pub. Date: Mar. 9, 2023

(54) METABOLITE BIOMARKER PROFILE AND METHOD OF USE TO DIAGNOSE PULMONARY ARTERIAL HYPERTENSION (PAH)

(71) Applicant: ARIZONA BOARD OF REGENTS ON BEHALF OF THE UNIVERSITY OF ARIZONA, Tucson, AZ (US)

(72) Inventors: Ruslan Rafikov, Tucson, AZ (US); Olga Rafikova, Tucson, AZ (US)

(21) Appl. No.: 17/792,902

(22) PCT Filed: Jan. 14, 2021
(86) PCT No.: PCT/US21/13405

§ 371 Date: Jul. 14, 2022

Related U.S. Application Data
(60) Provisional application No. 62/960,951, filed on Jan. 14, 2020.

(51) Int. Cl. G16H 50/20; G16B 25/00; G16B 40/20

(52) U.S. Cl. CPC G16H 50/20; G16B 25/00; G16B 40/20

(57) ABSTRACT

The present invention features a method comprising a metabolomic biomarker panel representing a metabolomic profile/fingerprint and methods of applying the profile to diagnose, monitor, and guide treatment for PAH. The profile comprises a unique panel of 36 metabolomic biomarkers/metabolites detected in plasma and/or urine obtained from the patient. The present invention allows for identification of patients with PAH in early-stage disease, before the condition has progressed sufficiently to produce clinical symptoms, and uniquely distinguishes PAH from pulmonary hypertension due to type 2 Diabetes Mellitus (DM) and/or left heart disease. The present invention allows for pre-screening of patients to identify PAH at the asymptomatic stage or help to minimize the time for PAH diagnosis after initial symptom onset.

### CROSS-REFERENCES TO RELATED APPLICATIONS

This application claims benefit of U.S. Provisional Application No. 62/960,951 filed Jan. 14, 2020, the specification(s) of which is/are incorporated herein in their entirety by reference.

### STATEMENT REGARDING FEDERALLY SPONSORED RESEARCH OR DEVELOPMENT

This invention was made with government support under Grant Nos. R01 HL132918 and R01 HL133085 awarded by National Institutes of Health. The government has certain rights in the invention.

### BACKGROUND OF THE INVENTION

Pulmonary arterial hypertension (PAH), a condition in which elevated pressure is found in the pulmonary artery, is an incurable, progressive disorder, and early diagnosis and treatment of PAH are associated with increased survival. However, due to the asymptomatic nature of PAH development as well as PAH's symptomatic similarity to hypertension from metabolic disorders (e.g., type 2 Diabetes Mellitus, DM) or left heart disease, there are severe delays in PAH diagnosis. In most cases, therapy starts only at an advanced stage of the disease and if it is not successfully treated, it can lead to right ventricular hypertrophy and right-sided heart failure. As such, there is a critical need to improve diagnostic approaches for diagnosis of PAH in its early stages and differentiation of similar pathologies.

### FIELD OF INVENTION

The present invention relates to diagnosing and monitoring PAH in a patient. The present invention features an in vitro diagnostic (IVD) tool comprising a metabolomic biomarker panel representing a profile or fingerprint and methods of using the profile or fingerprint for diagnosing and monitoring PAH; the profile comprises a unique panel of 36 biomarkers (e.g., a fingerprint) that can be detected in easily obtained specimens from a patient, for example, plasma, serum and/or urine. Without wishing to limit the present invention to any particular theory or mechanism, it is believed that metabolic reprogramming occurs early in the development of the disease and foreshadows pathophysiological changes. The present invention assesses a profile of circulating metabolites that uniquely allows the identification of patients with PAH at an early stage of disease, before the condition has progressed sufficiently to produce clinical symptoms. The profile or fingerprint distinguishes PAH from hypertension due to type 2 Diabetes Mellitus (DM) and left heart disease. The present invention features a set of metabolites for early diagnosis of PAH comprising—oxalic acid, aminomalonate, pseudouridine, gluconic acid, isothreonic acid, 4-hydroxyphenylacetic acid, erythritol, uric acid, uridine diphosphate (UDP)-glucuronic acid, fumaric acid, focuse, aconitic acid, 2-deoxytetronic acid, pantothenic acid, indole-3-acetate, myo-inositol, 2-hydroxyvaleric acid, citric acid, ribonic acid, glycine, glutamic acid, creatinine, glucuronic acid, phosphate, indole-3-lactate, urea, 2-hydroxyglutaric acid, tryptophan, tyrosine, glutamine, lysine, histidine, N-acetylornithine, 2-hydroxybutanoic acid, alpha-ketoglutarate, and oxoproline The present invention can be used for pre-screening of patients either to identify PAH at the asymptomatic stage or help to minimize the time for PAH diagnosis after the initial symptom onset.

### BACKGROUND ART

Pulmonary arterial hypertension (PAH) induces a distinct signature of circulating metabolites. Several previous reports were focused on the metabolic profiling of plasma and lung samples from pre-clinical models and patients. However, these prior comparisons were derived from healthy controls as described herein. It is more important to identify the markers that distinct PAH from other pathologies, which may show alterations of similar metabolites described herein. Metabolic reprogramming and a significant component of cardiac changes in PAH could mask PAH among more abundant conditions, such as metabolic disorders or left heart diseases. The present invention differentiates the plasma metabolic profiles of patients with idiopathic PAH, patients with type 2 diabetes mellitus (DM) and patients with left heart diseases (Heart).

Patients with idiopathic pulmonary arterial hypertension (IPAH) are particularly likely to be diagnosed at a late stage of the disease because the symptoms are nonspecific; diagnosis requires complex or invasive tests. This has increased the need for better diagnostic tools.

To date, there are no inexpensive, non-invasive specific screening tools. An increase in pulmonary artery pressure can be estimated by transthoracic echocardiography. It is currently used for screening, but in patients with PH, it often over- or under-estimates pulmonary arterial pressure and cardiac output. Nevertheless, echocardiography is a generally preferred non-invasive imaging modality for PH screening. Right heart catheterization is a standard diagnostic method for diagnosis but is not suitable for screening because it is invasive. Early recognition of the disease is a high priority and additional diagnostic and non-invasive screening tools need to be developed.

Biomarkers specifically indicating disease, disease stage, and treatment response to a particular treatment are ideal tools for optimization of pulmonary hypertension management. In addition, monitoring of pulmonary hypertension via biomarkers is important to better determine the urgency of lung transplantation in patients with diseases that are difficult to treat.

The present invention uses targeted metabolomics to quantify primary plasma metabolites or biomarkers, including carbohydrates, amino acids, and nucleotides for example by GC-TOF mass spectrometry. The present invention shows that the plasma metabolomics profile is distinctly different in patients with PAH compared to diabetic patients or patients with left heart diseases. Therefore, the unique plasma metabolome described herein has a strong potential to serve as a diagnostic tool.

The present invention shows that 36 unique metabolites are significantly altered in PAH patients compared to either diabetics or left heart patients or healthy people (Table 1) and could be used as a fingerprint for PAH. These unique metabolites can be classified into a few major groups comprising mitochondrial-derived metabolites, carbohydrates, myo-inositol and its derivatives, and metabolites that are indicative of damage known to be directly associated with PAH or an altered gut microbiome. Of these 36 metabolites, a minimum of 5 metabolites are critical for diagnosis of PAH and to differentiate PAH from DM or left heart disease.

TABLE 1
Metabolites that are significantly different in PAH cohort vs. heart or DM cohorts (a minimum
of five (5) metabolites critical for the diagnosis for PAH and to differentiate PAH from DM or
left heart disease). A fold change less than 1.0 in the healthy control indicates that the metabolite
is decreased in PAH compared to the compared cohort. A fold change greater than 1.0 in the
healthy control indicates that the metabolite is increased in PAH compared to the compared cohort.

| | Fold | | Fold | | Fold | |
|---|---|---|---|---|---|---|
| Metabolites | Heart | p-value | DM | p-value | Healthy | p-value |
| oxalic acid | 45.23 | 4.35 × 10⁻⁵ | 82.10 | 2.24 × 10⁻⁸ | 156.36 | 1.69 × 10⁻⁸ |
| aminomalonate | 3.00 | 6.52 × 10⁻⁵ | 2.75 | 6.12 × 10⁻³ | 6.60 | 2.66 × 10⁻⁸ |
| pseudouridine | 2.62 | 2.31 × 10⁻⁴ | 3.07 | 8.86 × 10⁻⁵ | 2.44 | 3.04 × 10⁻³ |
| gluconic acid | 2.35 | 6.25 × 10⁻⁸ | 1.55 | 1.33 × 10⁻² | 2.52 | 1.11 × 10⁻² |
| isothreonic acid | 2.05 | 1.31 × 10⁻⁵ | 2.09 | 9.85 × 10⁻⁴ | | |
| 4-hydroxyphenylacetic acid | 2.02 | 1.57 × 10⁻² | 2.24 | 2.77 × 10⁻³ | | |
| erythritol | 2.01 | 3.16 × 10⁻³ | 2.83 | 1.56 × 10⁻³ | 0.10 | 7.27 × 10⁻² |
| uric acid | 1.99 | 2.28 × 10⁻³ | 1.79 | 4.85 × 10⁻² | 4.12 | 7.98 × 10⁻⁷ |
| UDP-glucuronic acid | 1.98 | 4.45 × 10⁻³ | 4.49 | 2.77 × 10⁻⁴ | 4.49 | 3.08 × 10⁻¹ |
| fumaric acid | 1.95 | 3.81 × 10⁻⁵ | 1.37 | 1.16 × 10⁻² | 0.77 | 4.94 × 10⁻⁵ |
| focuse | 1.86 | 1.68 × 10⁻³ | 1.68 | 3.40 × 10⁻³ | | |
| aconitic acid | 1.86 | 5.21 × 10⁻⁵ | 3.14 | 1.68 × 10⁻⁹ | 4.64 | 3.57 × 10⁻¹ |
| 2-deoxytetronic acid | 1.78 | 1.36 × 10⁻² | 2.17 | 7.04 × 10⁻⁵ | 2.53 | 3.86 × 10⁻¹ |
| pantothenic acid | 1.75 | 7.33 × 10⁻³ | 1.69 | 8.60 × 10⁻⁴ | | |
| indole-3-acetate | 1.59 | 9.92 × 10⁻³ | 2.06 | 1.16 × 10⁻³ | | |
| myo-inositol | 1.50 | 2.52 × 10⁻² | 1.65 | 2.32 × 10⁻² | 2.33 | 9.44 × 10⁻³ |
| 2-hydroxyvaleric acid | 1.48 | 3.27 × 10⁻² | 2.12 | 5.16 × 10⁻³ | | |
| citric acid | 1.48 | 2.97 × 10⁻² | 2.01 | 1.81 × 10⁻⁴ | 1.96 | 1.34 × 10⁻⁵ |
| ribonic acid | 1.48 | 6.03 × 10⁻⁴ | 2.61 | 5.75 × 10⁻⁴ | | |
| glycine | 1.44 | 5.18 × 10⁻² | 1.41 | 4.17 × 10⁻² | | |
| glutamic acid | 1.40 | 4.91 × 10⁻² | 0.68 | 3.59 × 10⁻² | 1.74 | 4.02 × 10⁻² |
| creatinine | 1.38 | 1.55 × 10⁻² | 1.58 | 8.82 × 10⁻⁴ | | |
| glucuronic acid | 1.37 | 8.21 × 10⁻⁴ | 2.66 | 1.77 × 10⁻⁴ | | |
| phosphate | 1.28 | 4.56 × 10⁻² | 4.54 | 2.09 × 10⁻¹¹ | 0.23 | 1.27 × 10⁻¹⁷ |
| indole-3-lactate | 1.26 | 3.59 × 10⁻² | 1.85 | 4.37 × 10⁻⁶ | | |
| urea | 1.21 | 2.91 × 10⁻² | 1.47 | 2.49 × 10⁻² | | |
| 2-hydroxyglutaric acid | 0.78 | 2.88 × 10⁻² | 1.55 | 2.80 × 10⁻⁴ | | |
| tryptophan | 0.76 | 4.66 × 10⁻³ | 0.51 | 7.55 × 10⁻⁸ | | |
| tyrosine | 0.74 | 7.99 × 10⁻³ | 0.67 | 3.43 × 10⁻⁵ | | |
| glutamine | 0.73 | 4.99 × 10⁻³ | 0.71 | 2.97 × 10⁻² | | |
| lysine | 0.72 | 4.38 × 10⁻⁴ | 0.56 | 2.20 × 10⁻⁶ | | |
| histidine | 0.51 | 1.34 × 10⁻⁵ | 0.45 | 8.65 × 10⁻⁷ | | |
| N-acetylornithine | | | | | 7.56 | 4.14 × 10⁻⁶ |
| 2-hydroxybutanoic acid | | | | | 5.26 | 3.89 × 10⁻⁵ |
| alpha-ketoglutarate | | | | | 3.19 | 8.73 × 10⁻⁵ |
| oxoproline | | | | | 1.74 | 3.31 × 10⁻⁷ |

The present invention shows the value of metabolic profiling as a new diagnostic tool and provides subsequent research with a specific set of preselected metabolites that could serve as a fingerprint of PAH. The present invention also highlights the potential value of the particular metabolites in dissecting the pathogenesis of PAH.

### BRIEF SUMMARY OF THE INVENTION

It is an objective of the present invention to provide diagnostic and monitoring and treatment methods for PAH that allow for early diagnosis of PAH and distinction from DM and/or left heart disease, as specified in the independent claims. Embodiments of the invention are given in the dependent claims. Embodiments of the present invention can be freely combined with each other if they are not mutually exclusive.

One of the unique and inventive technical features of the present invention is using a profile or panel (fingerprint) of 5 out of 36 metabolites for early diagnosis of PAH. Without wishing to limit the invention to any theory or mechanism, it is believed that the technical feature of the present invention advantageously provides for differentiating PAH from DM and/or left heart disease. None of the presently known prior references or work has the unique inventive technical feature of the present invention.

Furthermore, the prior references teach away from the present invention. For example, metabolites described herein are also present and/or altered in similar PH pathologies from metabolic disorders (e.g., DM; left heart disease) and it would be counterintuitive or not obvious that these select metabolites can differentiate PAH from DM and/or left heart disease.

Furthermore, the inventive technical feature of the present invention contributed to a surprising result. For example, the inventors of the present invention surprisingly found that the determination of the level of five or more of the aforementioned 36 metabolites of a patient-derived biological sample enables diagnosis of PAH and differentiation from DM and left heart disease even though these metabolites are also altered in DM and left heart disease.

The present invention features a computer-implemented method for diagnosing a subject with a disease. In some embodiments, the method comprises inputting expression data of a panel of metabolic biomarkers in a biological sample obtained from the subject. In other embodiments, the method comprises determining whether expression of the metabolic biomarkers in the biological sample obtained from the subject is indicative of a disease using a computer system programmed with a trained machine learning classifier for distinguishing subjects with different diseases and without disease. In some embodiments, the machine learning classifier has been trained using expression data of a panel of metabolic biomarkers from subjects having disease and from control subjects that do not have disease. In some embodiments, the method comprises diagnosing the subject. In some embodiments, the expression data of the panel of metabolic biomarkers in the biological sample obtained from the subject is correlated by the computer system to be indicative of disease; and where the diagnostic accuracy is at least 90%. In other embodiments, the panel of metabolic biomarkers comprises at least 5 metabolites selected from a group consisting of oxalic acid, aminomalonate, pseudouridine, gluconic acid, isothreonic acid, 4-hydroxyphenylacetic acid, erythritol, uric acid, UDP-glucuronic acid, fumaric acid, focuse, aconitic acid, 2-deoxytetronic acid, pantothenic acid, indole-3-acetate, myo-inositol, 2-hydroxyvaleric acid, citric acid, ribonic acid, glycine, glutamic acid, creatinine, glucuronic acid, phosphate, indole-3-lactate, urea, 2-hydroxyglutaric acid, tryptophan, tyrosine, glutamine, lysine, histidine, N-acetylornithine, 2-hydroxybutanoic acid, alpha-ketoglutarate, oxoproline. In further embodiments, the metabolites may further comprise adenosine-5-monophosphate, 3-phenyllactic acid, pyrophosphate, maltotriose, glucose-1-phosphate, myristic acid, docosahexaenoic acid, aspartic acid, tocopherol gamma, 5-methoxytryptamine, arachidic acid, cystine, adipic acid, 3-hydroxybutyric acid, cholesterone, 2,4-diaminobutyric acid, and 3-aminoisobutyric acid

The present invention may further feature a method of treating a subject with pulmonary arterial hypertension (PAH). In some embodiments, the method comprises determining the expression data of a panel of metabolic biomarkers in a biological sample obtained from the subject. In some embodiments, the method comprises treating PAH in a subject identified as having differing levels of the panel of metabolic biomarkers when compared to the levels of said metabolic biomarkers in a normal subject. In other embodiments, the panel of metabolic biomarkers comprises oxalic acid, aminomalonate, pseudouridine, gluconic acid, erythritol, uric acid, UDP-glucuronic acid, fumaric acid, aconitic acid, 2-deoxytetronic acid, myo-inositol, citric acid, glutamic acid, phosphate, N-acetylornithine, 2-hydroxybutanoic acid, alpha-ketoglutarate, and oxoproline. In further embodiments, the subject with PAH has a higher level of oxalic acid, aminomalonate, pseudouridine, gluconic acid, uric acid, UDP-glucuronic acid, aconitic acid, 2-deoxytetronic acid, myo-inositol, citric acid. glutamic acid. N-acetylornithine, 2-hydroxybutanoic acid, alpha-ketoglutarate, oxoproline than the normal subject. In other embodiments, the subject with PAH has a lower level of erythritol, fumaric acid, and phosphate than the normal subject.

The present invention may feature a non-transitory, computer-readable medium having computer executable instructions for causing a processor to execute a method for diagnosing a subject with a disease. In some embodiments, the method comprises determining whether expression data of a panel of metabolic biomarkers in a biological sample obtained from the subject is indicative of the disease using a trained machine learning classifier for distinguishing subjects with different diseases and without disease. In some embodiments, the machine learning classifier has been trained using expression data of a panel of metabolic biomarkers from subjects having the disease and from control subjects that do not have disease. In some embodiments, the method comprises diagnosing the subject if the expression data is correlated to be indicative of the disease. In some embodiments, the diagnostic accuracy is at least 90%. In other embodiments, the panel of metabolic biomarkers comprises at least 5 metabolites selected from a group consisting of oxalic acid, aminomalonate, pseudouridine, gluconic acid, isothreonic acid, 4-hydroxyphenylacetic acid, erythritol, uric acid, UDP-glucuronic acid, fumaric acid, focuse, aconitic acid, 2-deoxytetronic acid, pantothenic acid, indole-3-acetate, myo-inositol, 2-hydroxyvaleric acid, citric acid, ribonic acid, glycine, glutamic acid, creatinine, glucuronic acid, phosphate, indole-3-lactate, urea, 2-hydroxyglutaric acid, tryptophan, tyrosine, glutamine, lysine, histidine, N-acetylornithine, 2-hydroxybutanoic acid, alpha-ketoglutarate, and oxoproline.

The present invention may also feature a kit for diagnosing a subject with a disease. In some embodiments, the kit comprises one or more reference metabolic biomarker panels (see Table 1, 2, 3). In other embodiments, the kit comprises a non-transitory, computer-readable medium described herein. In some embodiments, expression data of a panel of metabolic biomarkers in a biological sample obtained from the subject is inputted into a computer that executes the computer executable instructions of the non-transitory, computer-readable medium. In other embodiments, a subject is diagnosed with disease when the expression data of the panel of metabolic biomarkers in the biological subject is correlated with the one or more reference metabolic biomarker panels by the computer to be indicative of disease and where the diagnostic accuracy is at least 90%. In further embodiments, the panel of metabolic biomarkers comprises at least 5 metabolites selected from a group consisting of oxalic acid, aminomalonate, pseudouridine, gluconic acid, isothreonic acid, 4-hydroxyphenylacetic acid, erythritol, uric acid, UDP-glucuronic acid, fumaric acid, focuse, aconitic acid, 2-deoxytetronic acid, pantothenic acid, indole acetate, myo-inositol, 2-hydroxyvaleric acid, citric acid, ribonic acid, glycine, glutamic acid, creatinine, glucuronic acid, phosphate, indole-3-lactate, urea, 2-hydroxyglutaric acid, tryptophan, tyrosine, glutamine, lysine, histidine, N-acetylornithine, 2-hydroxybutanoic acid, alpha-ketoglutarate, and oxoproline. In further embodiments, the metabolites may further comprise adenosine-5-monophosphate, 3-phenyllactic acid, pyrophosphate, maltotriose, glucose-1-phosphate, myristic acid, docosahexaenoic acid, aspartic acid, tocopherol gamma, 5-methoxytryptamine, arachidic acid, cystine, adipic acid, 3-hydroxybutyric acid, cholesterone, 2,4-diaminobutyric acid, and 3-aminoisobutyric acid.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A and 1B show metabolic profiling of plasma of PAH patients (n=11) and comparison with diabetes mellitus (DM, n=12) and left heart disease (Heart, n=11) patients. These data indicate significantly (p<0.05) altered metabolites in PAH versus Heart and PAH versus DM analysis. FIG. 1A shows heat maps and FIG. 1B shows principal component analysis (PCA) showing the clustering of the PAH group that could differentiate PAH patients from DM and Heart by circulating metabolites.

FIG. 2A shows an analysis of two independent runs of plasma metabolites from PAH patients (n=11). Circles indicate the same patient in two experiments. Data showed good reproducibility.

FIG. 2B shows a comparison of PAH patients (n=11) with diabetes mellitus (DM, n=12) and left heart disease (Heart, n=11) patients altogether with only eleven selected metabolites that give maximal separation of the clustering analysis. Ellipses indicate the area of 0.95 probability that metabolic profiling of the patient from the same group will be inside an ellipse. Based on analysis of 11 preselected metabolites, the PAH group appears to be significantly (p<0.05) separated from either DM or Heart patients.

FIG. 3 shows circulating metabolites distinguish PAH patients from other diseases. FIG. 3 shows principal component analysis (PCA) of Health and PAH cohorts. Profiling of circulating metabolites was focused on eleven metabolites that significantly distinguish PAH patients from the healthy subjects. Ellipses indicate 95% confidence. By monitoring the Panel #1 metabolites (Table 1), healthy individuals are efficiently separated from PAH patients (FIG. 3). Ellipses show 95% confidence that the next examined person will fall inside the ellipse corresponding to the diagnosis. Notably, the PAH cohort consisted of two geographical locations (the University of Colorado and University of Arizona cohorts), which confirm that patients' geographical affiliation does not affect the test outcome

FIG. 4 shows early PAH detection using Panel #1 (Table 1). FIG. 4 shows a PCA in which there is an overlap between the mild PAH (mPAP 20-25 mmHg, diamonds), advanced PAH (mPAP>45 mmHg, cicles), and treatment naïve group (triangles). This result indicates that metabolically all groups are similar and could be efficiently identified regardless of the disease stage or treatment approach. Cohorts of patients were examined with early/mild PAH with borderline mean Pulmonary Arterial Pressure (mPAP=20-25 mmHg), with advanced PAH (mPAP>45 mmHg), and treatment naïve patients, who did not receive treatments. The data indicate that mild and advanced PAH were indistinguishable. Thus, Panel #1 (Table 1) can diagnose PAH at the early stage with the same accuracy as in advanced PAH. This highlights the test capability to effectively evaluate PAH's mildest form (mPAP=20-25 mmHg), which has recently been considered the upper limit of the normal. Furthermore, the complex therapeutic scheme, which could vary from patient to patient, does not affect the ability of Panel #1 (Table 1) to diagnose the PAH since we see no differences between the treated and treatment-naïve patient groups.

FIG. 5A-5B shows that machine learning/deep learning (ML/DL) algorithms can be used for metabolic pattern recognition. Machine learning algorithms are robust tools for recognizing a variety of patterns, including metabolic panels. The preliminary studies tested whether ML/DL classification approaches can be trained to label “unknown” samples with high precision. For this purpose, Panel #1 was used for the logistic regression algorithm training. After training, the algorithm randomly picked a sample and marked it as unknown (cross-validation), and predicted the disease. The confusion matrix shown in FIG. 5A indicates ML predictions for PAH patients vs. Healthy controls indication—100% accuracy. FIG. 5B shows the confusion matrix for predicting diabetics (d), left heart disease (h), and pulmonary hypertension (p), and Σ- indicates the number of “unknown samples” attempts. Based on these data, the ML/DL approach shows no confusion in diagnosing PAH patients using panels of circulating metabolites.

### DETAILED DESCRIPTION OF THE INVENTION

Any methods, devices and materials similar or equivalent to those described herein can be used in the practice of this invention. The following definitions are provided to facilitate understanding of certain terms used frequently herein and are not meant to limit the scope of the present disclosure. In the event that there is a plurality of definitions for a term herein, those in this section prevail unless stated otherwise. Headings used herein are for organizational purposes only and in no way limit the invention described herein.

As used herein, the term “pulmonary hypertension (PH) refers to a disease characterized by an increase of blood pressure in the pulmonary artery. Pulmonary hypertension is determined as mean pulmonary artery pressure (mPAP) 20 mm Hg at rest measured by right heart catheterization. The increase in pulmonary arterial blood pressure may lead to shortness of breath, dizziness, fainting, leg swelling, and other symptoms. Pulmonary hypertension can be a severe disease with a markedly decreased exercise tolerance. The disease may be hereditary. According to the most recent classification of the World Health Organization (WHO), pulmonary hypertension can be one of the following different types: pulmonary arterial hypertension (PAH), pulmonary hypertension due to left heart disease, pulmonary hypertension due to lung disease and/or hypoxia (e. g. pulmonary hypertension (PH) due to chronic obstructive pulmonary disease (COPD) or PH due to interstitial lung disease (ILD), chronic thromboembolic pulmonary hypertension (CTEPH) or pulmonary hypertension with unclear multifactorial mechanisms (group 5).

As used herein, the term “pulmonary arterial hypertension (PAH)” refers to a disease characterized by an increase of blood pressure in the pulmonary artery where underlying causes for example left heart disease, lung disease, CTEPH, and group 5 diseases have been excluded. Pulmonary arterial hypertension includes a number of subgroups. In particular, it encompasses idiopathic pulmonary arterial hypertension (IPAH), hereditary PAH, and associated PAH (APAH) which may be associated to drug or toxins, connective tissue diseases, HIV infection, portal hypertension.

As used herein, PAH and PH may be used interchangeably.

As used herein, “administering” and the like refer to the act of physically delivering a composition or other therapy (e.g. a treatment for PAH) described herein into a subject by such routes as oral, mucosal, topical, transdermal, suppository, intravenous, parenteral, intraperitoneal, intramuscular, intralesional, intrathecal, intranasal or subcutaneous administration. Parenteral administration includes intravenous, intramuscular, intra-arterial, intradermal, subcutaneous, intraperitoneal, intraventricular, and intracranial administration. When a disease, disorder or condition, or a symptom thereof, is being treated, administration of the substance typically occurs after the onset of disease, disorder or condition or symptoms thereof. When a disease, disorder or condition, or symptoms thereof, are being prevented, administration of the substance typically occurs before the onset of the disease, disorder or condition or symptoms thereof.

As used herein, the terms “subject” and “patient” are used interchangeably. As used herein, a subject can be an animal (amphibian, reptile, avian, fish, or mammal) such as a non-primate (e.g., cows, pigs, horses, cats, dogs, rats, etc.) or a primate (e.g., monkey, ape and human). In specific embodiments, the subject is a human. In one embodiment, the subject is a mammal (e.g., a human, a dog) having a disease, disorder or condition described herein. In another embodiment, the subject is a mammal (e.g., a human, a dog) at risk of developing a disease, disorder or condition described herein. In certain instances, the term patient refers to a human under medical care or animals under veterinary care.

As used herein, the terms “normal subject or “healthy subject” or “control subject” may be used interchangeably and refers to a subject with no underlying chronic or acute disease conditions.

The terms “treating” or “treatment” refer to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being. Common treatments for PAH comprise administration of a drug, surgery, transplantation, exercise training, physical rehabilitation, and/or balloon angioplasty.

The term “effective amount” as used herein refers to the amount of a therapy or medication (e.g., treatment for PAH) which is sufficient to reduce and/or ameliorate the severity and/or duration of a given disease, disorder or condition and/or a symptom related thereto. This term also encompasses an amount necessary for the reduction or amelioration of the advancement or progression of a given disease (e.g., PAH), disorder or condition, reduction or amelioration of the recurrence, development or onset of a given disease, disorder or condition, and/or to improve or enhance the prophylactic or therapeutic effect(s) of another therapy. In some embodiments, “effective amount” as used herein also refers to the amount of therapy provided herein to achieve a specified result.

As used herein, and unless otherwise specified, the term “therapeutically effective amount” of a drug or intervention is an amount sufficient enough to provide a therapeutic benefit in the treatment or management of a PAH, or to delay or minimize one or more symptoms associated with the presence of PAH. A therapeutically effective amount of a treatment for PAH, means an amount of therapeutic agent, alone or in combination with other therapies, which provides a therapeutic benefit in the treatment or management of PAH. The term “therapeutically effective amount” can encompass an amount that improves overall therapy, reduces or avoids symptoms or causes of PAH, or enhances the therapeutic efficacy of another therapeutic agent.

A therapy is any protocol, method and/or agent that can be used in the prevention, management, treatment and/or amelioration of a given disease, disorder or condition. In certain embodiments, the terms “therapies” and “therapy” refer to a drug therapy, biological therapy, supportive therapy, radiation therapy, exercise therapy, and/or other therapies useful in the prevention, management, treatment and/or amelioration of a given disease, disorder or condition known to one of skill in the art such as medical personnel. Common therapies for PAH comprise administration of a drug, surgery, transplantation, exercise training, physical rehabilitation, and/or balloon angioplasty.

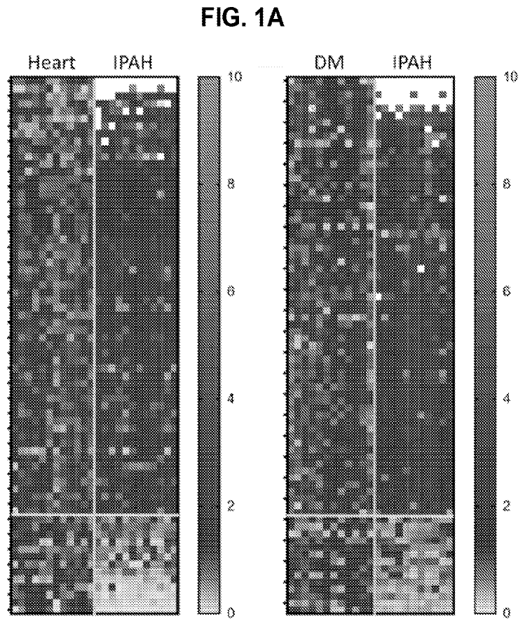

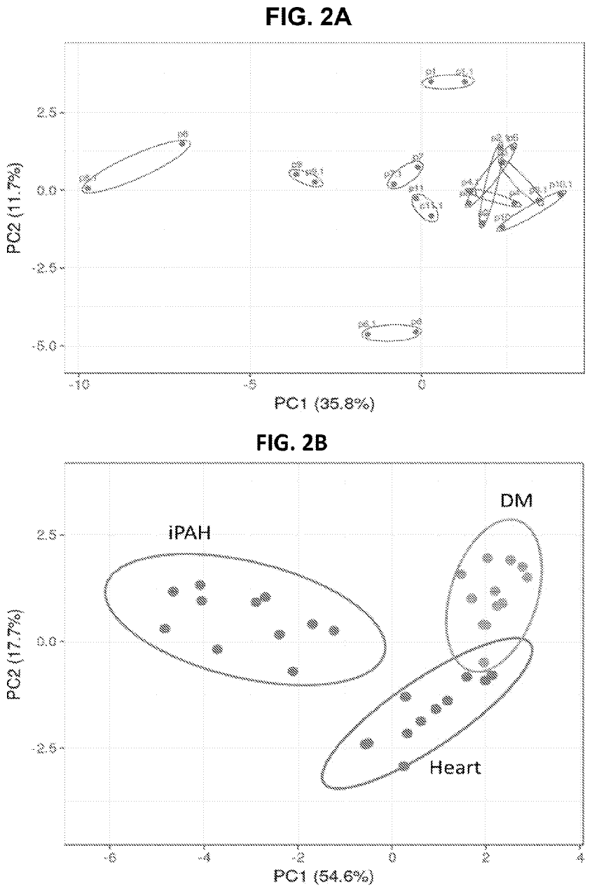

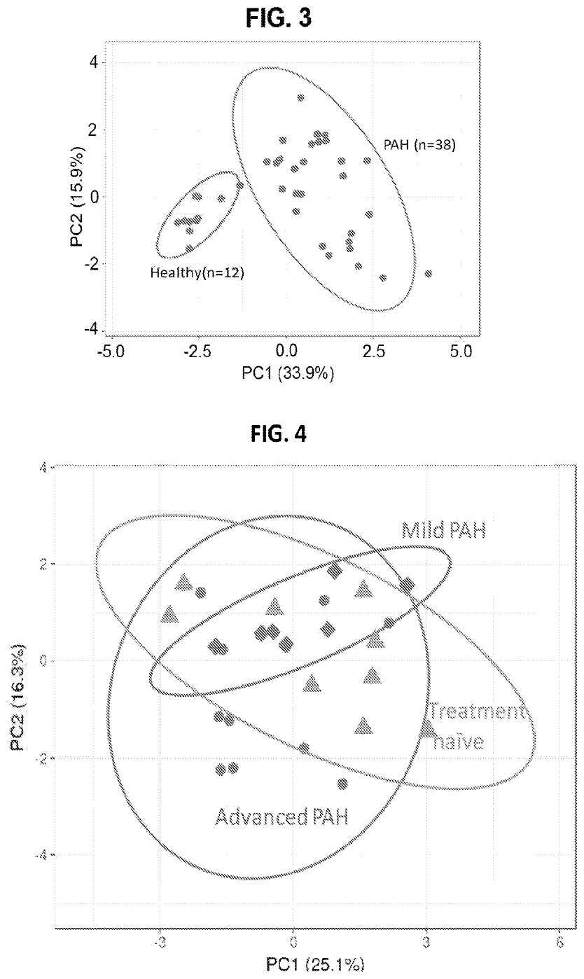

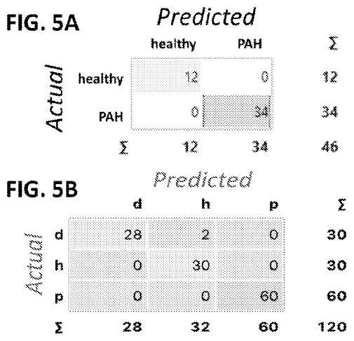

Referring now to FIGS. 1A, 1B, 2A, 2B, 3, 4, 5A and 5B the present invention features diagnostic, treatment and monitoring methods, and kits for PAH that allow for early diagnosis as well as monitoring of PAH and distinction of PAH from DM and/or left heart disease. The present invention utilizes targeted metabolomics to determine levels of metabolites, which are measured in a biological sample (e.g., plasma, urine, serum) using standard analytical techniques including GC-TOF mass spectrometry. The metabolites comprise oxalic acid, aminomalonate, pseudouridine, gluconic acid, isothreonic acid, 4-hydroxyphenylacetic acid, erythritol, uric acid, uridine diphosphate (UDP)-glucuronic acid, fumaric acid, focuse, aconitic acid, 2-deoxytetronic acid, pantothenic acid, indole-3-acetate, myo-inositol, 2-hydroxyvaleric acid, citric acid, ribonic acid, glycine, glutamic acid, creatinine, glucuronic acid, phosphate, indole-3-lactate, urea, 2-hydroxyglutaric acid, tryptophan, tyrosine, glutamine, lysine, histidine, N-acetylornithine, 2-hydroxybutanoic acid, alpha-ketoglutarate, and oxoproline. The metabolites may further comprise adenosine-5-monophosphate, 3-phenyllactic acid, pyrophosphate, maltotriose, glucose-1-phosphate, myristic acid, docosahexaenoic acid, aspartic acid, tocopherol gamma, 5-methoxytryptamine, arachidic acid, cystine, adipic acid, 3-hydroxybutyric acid, cholesterone, 2,4-diaminobutyric acid, and 3-aminoisobutyric acid. A metabolic profile is then determined based on the measured level of at least one metabolite. The profile is a metabolic fingerprint that distinguishes patients with PAH as compared to those with DM and/or left heart disease. This fingerprint can be used for diagnosis, monitoring, guiding treatment for patients having or suspected of having PAH.

The present invention features a computer-implemented method for diagnosing a subject with a disease. In some embodiments, the method comprises inputting expression data of a panel of metabolic biomarkers in a biological sample obtained from the subject. In other embodiments, the method comprises determining whether expression of the metabolic biomarkers in the biological sample obtained from the subject is indicative of a disease using a computer system programmed with a trained machine learning classifier for distinguishing subjects with different diseases and without disease. In some embodiments, the machine learning classifier has been trained using expression data of a panel of metabolic biomarkers from subjects having disease and from control subjects that do not have disease. In some embodiments, the method comprises diagnosing the subject. In some embodiments, the expression data of the panel of metabolic biomarkers in the biological sample obtained from the subject is correlated by the computer system to be indicative of disease; and where the diagnostic accuracy is at least 90%. In other embodiments, the panel of metabolic biomarkers comprises at least 5 metabolites selected from a group consisting of oxalic acid, aminomalonate, pseudouridine, gluconic acid, isothreonic acid, 4-hydroxyphenylacetic acid, erythritol, uric acid, UDP-glucuronic acid, fumaric acid, focuse, aconitic acid, 2-deoxytetronic acid, pantothenic acid, indole-3-acetate, myo-inositol, 2-hydroxyvaleric acid, citric acid, ribonic acid, glycine, glutamic acid, creatinine, glucuronic acid, phosphate, indole-3-lactate, urea, 2-hydroxyglutaric acid, tryptophan, tyrosine, glutamine, lysine, histidine, N-acetylornithine, 2-hydroxybutanoic acid, alpha-ketoglutarate, oxoproline. In further embodiments, the metabolites may further comprise adenosine-5-monophosphate, 3-phenyllactic acid, pyrophosphate, maltotriose, glucose-1-phosphate, myristic acid, docosahexaenoic acid, aspartic acid, tocopherol gamma, 5-methoxytryptamine, arachidic acid, cystine, adipic acid, 3-hydroxybutyric acid, cholesterone, 2,4-diaminobutyric acid, and 3-aminoisobutyric acid.

In some embodiments, the expression data of the panel of metabolic biomarkers is determined using standard clinical chemistry techniques, protein analytic techniques, nucleic acid techniques, and/or analytical techniques suitable for metabolite analysis (e.g., Gas chromatography—time of flight mass spectrometry (GC-TOF) mass spectrometry, liquid chromatography mass spectrometry (LCMS), Nuclear magnetic resonance (NMR)).

In some embodiments, the trained machine learning classifiers are the machine learning/deep learning algorithms including logistic regression, neural network, and other algorithms. As used herein, “a machine learning classifier” utilizes some training data to train algorithms to predict the class (a disease) with given input variables (expression data of metabolic biomarkers).

In some embodiments the present invention may include a processor in communication with various elements of hardware. In some embodiments, the processor includes one or more processors configured to implement a set of instructions corresponding to any of the methods disclosed herein. In other embodiments, the processor can be configured to implement a set of instructions (stored in memory of hardware or sub-system) to provide a correlation between the expression data and a particular disease. In other embodiments, a sub-system can include hardware and software capable of facilitating the processing of data generated by hardware, in conjunction with, or as a substitute for, the processing that is normally handled by the processor.

In some embodiments, the diagnostic accuracy of the computer system is 100%. In some embodiments, the diagnostic accuracy of the computer system is at least 99%. In some embodiments, the diagnostic accuracy of the computer system is at least 98%. In some embodiments, the diagnostic accuracy of the computer system is at least 95%. In some embodiments, the diagnostic accuracy of the computer system is at least 90%. In some embodiments, the diagnostic accuracy of the computer system is 85%. In some embodiments, the diagnostic accuracy of the computer system is at least 80%.

In other embodiments, the present invention utilizes metabolites comprising carbohydrates, amino acids, fatty acids and/or nucleotides and their derivatives.

In some embodiments, the present invention is for a patient who is asymptomatic or symptomatic for pulmonary hypertension, diabetes mellitus, heart disease, lung diseases, chronic liver, kidney diseases, cancers and other chronic or acute conditions characterized with metabolic changes in the mitochondria, alterations in glycolytic pathways, protein biosynthesis/oxidation, fatty acids biosynthesis/oxidations, etc. In preferred embodiments, the biological sample is easily obtained from the patient and can comprise plasma, serum, urine, lung fluids, lavage, etc.

In some embodiments, the metabolites are measured throughout time in biological samples obtained from subjects longitudinally throughout time. Non-limiting examples of longitudinal timing points comprise 1) an initial time comprising at time of no symptoms, at time of diagnosis, or at initial presentation of symptoms; 2) about weekly intervals post initial time; 3) about monthly intervals post initial time; and/or 4) about yearly intervals.

In some embodiment, the disease comprises pulmonary arterial hypertension (PAH), Diabetes Mellitus (DM), left heart disease, chronic obstructive pulmonary disease (COPD), interstitial lung disease (ILD), head and neck cancer, thyroid cancer or colon cancer. In some embodiments, the method is able to differentiate between PAH, DM, and left heart disease. In other embodiments, the metabolic biomarker panel is distinctly different in subjects with pulmonary arterial hypertension (PAH) compared to subjects with Diabetes Mellitus (DM) or left heart disease.

In some embodiments, the method may be used for pre-screening the subject either to identify pulmonary arterial hypertension (PAH) at the asymptomatic stage or to minimize the time for PAH diagnosis after the initial symptom onset. In other embodiments, the method is used for determining the severity of PAH in the patient. In further embodiments, the method discriminates between pulmonary arterial hypertension (PAH) and classical metabolic disorders, including diabetes mellitus (DM), or left heart diseases. In other embodiments, the method is used in subjects with other diseases comprising cancer, lung diseases, and systemic hypertension. In further embodiments, the method is used to guide treatment and/or care management of the subject diagnosed with pulmonary arterial hypertension (PAH).

In some embodiments, the subject does not display clinical symptoms (i.e., asymptomatic) or does display clinical symptoms (i.e., symptomatic) for pulmonary arterial hypertension (PAH), DM, left heart diseases, lung diseases, kidney and liver disease, cancers or other acute and chronic conditions characterized with metabolic changes, wherein symptoms comprise, shortness of breath, first noticeable with exercise, but as the disease progresses, even during rest; feeling tired (fatigue); swelling in the feet, legs, belly or neck; chest pain; and/or heart pounding (palpitations).

In some embodiments, a biological sample may comprise plasma, serum, or urine, obtained from the subject.

The present invention may further feature a method of treating a subject with pulmonary arterial hypertension (PAH). In some embodiments, the method comprises determining the expression data of a panel of metabolic biomarkers in a biological sample obtained from the subject. In some embodiments, the method comprises treating PAH in a subject identified as having differing levels of the panel of metabolic biomarkers when compared to the levels of said metabolic biomarkers in a normal subject. In other embodiments, the panel of metabolic biomarkers comprises oxalic acid, aminomalonate, pseudouridine, gluconic acid, erythritol, uric acid, UDP-glucuronic acid, fumaric acid, aconitic acid, 2-deoxytetronic acid, myo-inositol, citric acid, glutamic acid, phosphate, N-acetylornithine, 2-hydroxybutanoic acid, alpha-ketoglutarate, and oxoproline. In further embodiments, the subject with PAH has a higher level of oxalic acid, aminomalonate, pseudouridine, gluconic acid, uric acid, UDP-glucuronic acid, aconitic acid, 2-deoxytetronic acid, myo-inositol, citric acid, glutamic acid, N-acetylornithine, 2-hydroxybutanoic acid, alpha-ketoglutarate, oxoproline than the normal subject. In other embodiments, the subject with PAH has a lower level of erythritol, fumaric acid, and phosphate than the normal subject.

In some embodiments, the expression data of the panel of metabolic biomarkers is determined using standard clinical chemistry techniques, protein analytic techniques, nucleic acid techniques, and/or analytical techniques suitable for metabolite analysis (e.g., Gas chromatography—time of flight mass spectrometry (GC-TOF) mass spectrometry, liquid chromatography mass spectrometry (LCMS), Nuclear magnetic resonance (NMR)).

In some embodiments, the method is used for determining the severity of PAH in the subject. In other embodiments, the subject does not display clinical symptoms (i.e., asymptomatic) or does display clinical symptoms (i.e., symptomatic) for pulmonary arterial hypertension (PAH), DM, left heart diseases, lung diseases, kidney and liver disease, cancers or other acute and chronic conditions characterized with metabolic changes, wherein symptoms comprise, shortness of breath, first noticeable with exercise, but as the disease progresses, even during rest; feeling tired (fatigue); swelling in the feet, legs, belly or neck; chest pain; and/or heart pounding (palpitations). In some embodiments, the method is used to guide treatment and/or care management of the subject diagnosed with pulmonary arterial hypertension (PAH).

In some embodiments, the biological sample comprises plasma, serum, urine, obtained from the subject.

In some embodiments, the treatment of pulmonary hypertension comprises administration of a drug, surgery, transplantation, exercise training, physical rehabilitation, and/or balloon angioplasty.

The present invention may feature a non-transitory, computer-readable medium having computer executable instructions for causing a processor to execute a method for diagnosing a subject with a disease. In some embodiments, the method comprises determining whether expression data of a panel of metabolic biomarkers in a biological sample obtained from the subject is indicative of the disease using a trained machine learning classifier for distinguishing subjects with different diseases and without disease. In some embodiments, the machine learning classifier has been trained using expression data of a panel of metabolic biomarkers from subjects having the disease and from control subjects that do not have disease. In some embodiments, the method comprises diagnosing the subject if the expression data is correlated to be indicative of the disease. In some embodiments, the diagnostic accuracy is at least 90%. In other embodiments, the panel of metabolic biomarkers comprises at least 5 metabolites selected from a group consisting of oxalic acid, aminomalonate, pseudouridine, gluconic acid, isothreonic acid, 4-hydroxyphenylacetic acid, erythritol, uric acid, UDP-glucuronic acid, fumaric acid, focuse, aconitic acid, 2-deoxytetronic acid, pantothenic acid, indole-3-acetate, myo-inositol, 2-hydroxyvaleric acid, citric acid, ribonic acid, glycine, glutamic acid, creatinine, glucuronic acid, phosphate, indole-3-lactate, urea, 2-hydroxyglutaric acid, tryptophan, tyrosine, glutamine, lysine, histidine, N-acetylornithine, 2-hydroxybutanoic acid, alpha-ketoglutarate, and oxoproline.

The present invention may also feature a kit for diagnosing a subject with a disease. In some embodiments, the kit comprises one or more reference metabolic biomarker panels (see Table 1, 2, 3). In other embodiments, the kit comprises a non-transitory, computer-readable medium described herein. In some embodiments, expression data of a panel of metabolic biomarkers in a biological sample obtained from the subject is inputted into a computer that executes the computer executable instructions of the non-transitory, computer-readable medium. In other embodiments, a subject is diagnosed with disease when the expression data of the panel of metabolic biomarkers in the biological subject is correlated with the one or more reference metabolic biomarker panels by the computer to be indicative of disease and where the diagnostic accuracy is at least 90%. In further embodiments, the panel of metabolic biomarkers comprises at least 5 metabolites selected from a group consisting of oxalic acid, aminomalonate, pseudouridine, gluconic acid, isothreonic acid, 4-hydroxyphenylacetic acid, erythritol, uric acid, UDP-glucuronic acid, fumaric acid, focuse, aconitic acid, 2-deoxytetronic acid, pantothenic acid, indole-3-acetate, myo-inositol, 2-hydroxyvaleric acid, citric acid, ribonic acid, glycine, glutamic acid, creatinine, glucuronic acid, phosphate, indole-3-lactate, urea, 2-hydroxyglutaric acid, tryptophan, tyrosine, glutamine, lysine, histidine, N-acetylornithine, 2-hydroxybutanoic acid, alpha-ketoglutarate, and oxoproline. In further embodiments, the metabolites may further comprise adenosine-5-monophosphate, 3-phenyllactic acid, pyrophosphate, maltotriose, glucose-1-phosphate, myristic acid, docosahexaenoic acid, aspartic acid, tocopherol gamma, 5-methoxytryptamine, arachidic acid, cystine, adipic acid, 3-hydroxybutyric acid, cholesterone, 2,4-diaminobutyric acid, and 3-aminoisobutyric acid.

In some embodiments, the expression data of the panel of metabolic biomarkers is determined using standard clinical chemistry techniques, protein analytic techniques, nucleic acid techniques, and/or analytical techniques suitable for metabolite analysis (e.g., Gas chromatography—time of flight mass spectrometry (GC-TOF) mass spectrometry, liquid chromatography mass spectrometry (LCMS), Nuclear magnetic resonance (NMR)).

In some embodiments, the metabolites are measured throughout time in biological samples obtained from subjects longitudinally throughout time. Non-limiting examples of longitudinal timing points comprise 1) an initial time comprising at time of no symptoms, at time of diagnosis, or at initial presentation of symptoms; 2) about weekly intervals post initial time; 3) about monthly intervals post initial time; and/or 4) about yearly intervals.

In some embodiment, the disease comprises pulmonary arterial hypertension (PAH), Diabetes Mellitus (DM), left heart disease, chronic obstructive pulmonary disease (COPD), interstitial lung disease (ILD), head and neck cancer, thyroid cancer or colon cancer. In some embodiments, the method is able to differentiate between PAH, DM, and left heart disease. In other embodiments, the metabolic biomarker panel is distinctly different in subjects with pulmonary arterial hypertension (PAH) compared to subjects with Diabetes Mellitus (DM) or left heart disease.

In some embodiments, the kit may be used for pre-screening the subject either to identify pulmonary arterial hypertension (PAH) at the asymptomatic stage or to minimize the time for PAH diagnosis after the initial symptom onset. In other embodiments, the kit is used for determining the severity of PAH in the patient. In further embodiments, the kit discriminates between pulmonary arterial hypertension (PAH) and classical metabolic disorders, including diabetes mellitus (DM), or left heart diseases. In other embodiments, the kit is used in subjects with other diseases comprising cancer, lung diseases, and systemic hypertension. In further embodiments, the method is used to guide treatment and/or care management of the subject diagnosed with pulmonary arterial hypertension (PAH).

In some embodiments, the subject does not display clinical symptoms (i.e., asymptomatic) or does display clinical symptoms (i.e., symptomatic) for pulmonary arterial hypertension (PAH), DM, left heart diseases, lung diseases, kidney and liver disease, cancers or other acute and chronic conditions characterized with metabolic changes, wherein symptoms comprise, shortness of breath, first noticeable with exercise, but as the disease progresses, even during rest; feeling tired (fatigue); swelling in the feet, legs, belly or neck; chest pain; and/or heart pounding (palpitations).

In other embodiments, the subject is asymptomatic or symptomatic for PAH, DM, left heart disease, wherein an asymptomatic patient does not display clinical symptoms of PAH, DM, left heart disease and asymptomatic patient displays clinical symptoms of PAH, DM, left heart disease comprising shortness of breath, first noticeable with exercise, but as the disease progresses, even during rest; feeling tired (fatigue); swelling in the feet, legs, belly or neck; chest pain; and/or heart pounding (palpitations).

In other embodiments, the 36 metabolites (Table 1) comprise mitochondrial-derived metabolites, carbohydrates, metabolites of myo-inositol and its derivatives, and/or metabolites that are indicative of damage known to be directly associated with PAH or an altered gut microbiome, and/or metabolites associated with PAH pathogenesis. Non-limiting examples of mitochondrial-derived metabolites comprise tricarboxylic acid (TCA) metabolites and their derivatives, including oxalic acid.

In preferred embodiments, the metabolomic biomarker panel is distinctly different in patients with PAH compared to diabetic patients or patients with left heart diseases. In other embodiments, the tools, methods, and kits described herein using the metabolomic profile or fingerprint is used for 1) pre-screening of patients either to identify PAH at the asymptomatic stage or to minimize the time for PAH diagnosis after the initial symptom onset; 2) determining the severity of PAH in a patient; 3) discriminating between PAH and classical metabolic disorders (e.g., DM) or left heart diseases that often mimic the symptoms of right heart disease; 4) patient cohorts with other diseases comprising cancer, lung diseases, and systemic hypertension; and 5) treatment and/or care management of a patient suspected of having or has PAH.

In some embodiments, 36 unique plasma metabolites or markers differentiate patients with PAH, DM, and left heart diseases. Of the 36 metabolites that are significantly altered in PAH as compared to diabetes mellitus and left heart diseases.

In some embodiments, symptomatic patients comprise patients with clinical symptoms of pulmonary stress, shortness of breath, first noticeable with exercise, but as the disease progresses, even during rest; feeling tired (fatigue); swelling in the feet, legs, belly or neck; chest pain; and/or heart pounding (palpitations). In other embodiments, asymptomatic patients do not overtly display the aforementioned clinical symptoms.

TABLE 2
Metabolites that are significantly different between
pulmonary hypertension, COPD and ILD cohorts.

| | Fold ILD | p-value | Fold COPD | p-value |
|---|---|---|---|---|
| adenosine-5-monophosphate | 0.322291 | 0.000924 | 0.38641893 | 0.008464 |
| 3-phenyllactic acid | 0.492135 | 0.001583 | 1.09598575 | 0.659976 |
| pyrophosphate | 0.503944 | 0.002213 | 0.5896616 | 0.009202 |
| uric acid | 1.938433 | 0.007498 | 1.52389566 | 0.042332 |
| maltotriose | 0.367153 | 0.012609 | 0.41858632 | 0.020487 |
| glucose-1-phosphate | 1.811182 | 0.015604 | 1.17356234 | 0.366675 |
| myristic acid | 1.945851 | 0.025099 | 1.70557262 | 0.043406 |
| docosahexaenoic acid | 2.030901 | 0.040692 | 1.53074952 | 0.136565 |
| aspartic acid | 0.837658 | 0.25527 | 0.6776052 | 0.010062 |
| myo-inositol | 0.853897 | 0.26947 | 0.69733062 | 0.012334 |
| tocopherol gamma- | 1.296965 | 0.278877 | 1.88540879 | 0.029407 |
| 5-methoxytryptamine | 0.669009 | 0.441173 | 1.94907759 | 0.414254 |
| arachidic acid | 1.099185 | 0.474805 | 1.3291263 | 0.049677 |

TABLE 3
Metabolites that are significantly different in colon,
thyroid, and Head and neck cancer cohort.

| | | | p-value |
|---|---|---|---|
| | p-value | p-value | Head& |
| | Colon | Thyroid | Neck |
| cystine | 1.65E−06 | 9.19E−03 | 2.73E−02 |
| adenosine-5-monophosphate | 2.25E−05 | 7.45E−01 | 3.88E−02 |
| adipic acid | 2.34E−05 | 2.28E−01 | 7.00E−01 |
| creatinine | 1.45E−04 | 6.22E−02 | 6.99E−01 |
| 3-hydroxybutyric acid | 1.59E−04 | 8.00E−02 | 6.32E−02 |
| cholesterone | 4.77E−04 | 2.33E−05 | 7.15E−06 |
| 2,4-diaminobutyric acid | 1.23E−03 | 7.46E−03 | 1.06E−04 |
| aminomalonate | 4.14E−03 | 4.99E−08 | 9.27E−10 |
| oxalic acid | 1.01E−02 | 4.80E−10 | 7.24E−08 |
| 3-aminoisobutyric acid | 2.09E−02 | 8.13E−02 | 9.62E−01 |
| N-acetylornithine | 4.67E−01 | 2.47E−08 | 3.76E−10 |

The present invention also features an in vitro method for diagnosing, monitoring, or treating a patient suffering pulmonary arterial hypertension (PAH) in a patient. In preferred embodiments, the method comprises first obtaining a biological sample from the patient. Non-limiting examples of a biological sample comprise an easily obtained sample from a patient, e.g., plasma, serum, blood, and/or urine. Targeted metabolomics is then performed to determine levels of metabolites, which are measured in the biological sample using standard analytical techniques including GC-TOF mass spectrometry. The metabolites comprise oxalic acid, aminomalonate, pseudouridine, gluconic acid, isothreonic acid, 4-hydroxyphenylacetic acid, erythritol, uric acid, uridine diphosphate (UDP)-glucuronic acid, fumaric acid, focuse, aconitic acid, 2-deoxytetronic acid, pantothenic acid, indole-3-acetate, myo-inositol, 2-hydroxyvaleric acid, citric acid, ribonic acid, glycine, glutamic acid, creatinine, glucuronic acid, phosphate, indole-3-lactate, urea, 2-hydroxyglutaric acid, tryptophan, tyrosine, glutamine, lysine, histidine, N-acetylornithine, 2-hydroxybutanoic acid, alpha-ketoglutarate, and oxoproline. The metabolites may further comprise adenosine-5-monophosphate, 3-phenyllactic acid, pyrophosphate, maltotriose, glucose-1-phosphate, myristic acid, docosahexaenoic acid, aspartic acid, tocopherol gamma, 5-methoxytryptamine, arachidic acid, cystine, adipic acid, 3-hydroxybutyric acid, cholesterone, 2,4-diaminobutyric acid, and 3-aminoisobutyric acid. In some embodiments, a metabolic profile is then determined based on the measured level of at least five metabolites. In some embodiments, the profile is a metabolic fingerprint that distinguishes patients with PAH as compared to those with Diabetes Mellitus (DM) and/or left heart disease.

In some embodiments, the patient is then diagnosed for PAH based on the metabolic profiled fingerprint that distinguishes patients with PAH as compared to those with Diabetes Mellitus (DM) and/or left heart disease. In other embodiments, the patient is then monitored throughout time for reduction, progression, or stable disease of PAH based on the metabolic profiled fingerprint that distinguishes patients with PAH as compared to those with DM and/or left heart disease. Non-limiting examples of longitudinal timepoints to measure the metabolites comprise at initial assessment for PAH, presentation of initial symptoms of PAH or diagnosis PAH, and then weekly, monthly, and/or yearly timepoints. In further embodiments, if the patient is diagnosed for PAH based on the metabolic profiled fingerprint that distinguishes patients with PAH as compared to those with Diabetes Mellitus (DM) and/or left heart disease. An effective amount of a therapeutic treatment is then administered based on the fingerprint to effectively treat the patient with PAH. The treatment of pulmonary hypertension/PAH may be selected from the group consisting of the administration of a drug, surgery, transplantation, exercise training, physical rehabilitation, and balloon angioplasty.

In some embodiments, the present invention includes a computer system that can execute the methods for diagnosing a disease as described herein. In some embodiments, the invention employs a computer device or computer-implemented method having one or more processors and at least one memory, the at least one memory storing non-transitory computer-readable instructions for execution by the one or more processors to cause the one or more processors to execute instructions (or stored data) in one or more modules. Alternatively, the instructions may be stored in a non-transitory computer-readable medium or computer-usable medium. In some embodiments, a computer system can include a desktop computer, a laptop computer, a tablet, or the like and can include digital electronic circuitry, firmware, hardware, memory, a computer storage medium, a computer program, a processor (including a programmed processor), or the like. The computing system may include a desktop computer with a screen and a tower.

The term “processor” encompasses all kinds of apparatus, devices, and machines for processing data, including by way of example a programmable microprocessor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures. The processor may include one or more processors of any type, such as central processing units (CPUs), graphics processing units (GPUs), special-purpose signal or image processors, field-programmable gate arrays (FPGAs), tensor processing units (TPUs), and so forth.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

Embodiments of the subject matter and the operations described herein can be implemented in digital electronic circuitry, or in computer software, firmware, or hardware, including the structures disclosed in this specification and their structural equivalents, or in combinations of one or more of them. Embodiments described in this specification can be implemented as one or more computer programs, i.e., one or more modules of computer program instructions, encoded on computer storage medium for execution by, or to control the operation of, data processing apparatus. Any of the modules described herein may include logic that is executed by the processor(s). “Logic,” as used herein, refers to any information having the form of instruction signals and/or data that may be applied to affect the operation of a processor. Software is an example of logic. Logic may be formed from signals stored on a computer-readable medium such as memory that, in an exemplary embodiment, may be a random access memory (RAM), read-only memories (ROM), erasable/electrically erasable programmable read-only memories (EPROMS/EEPROMS), flash memories, etc. Logic may also comprise digital and/or analog hardware circuits, for example, hardware circuits comprising logical AND, OR, XOR, NAND, NOR, and other logical operations. Logic: may be formed from combinations of software and hardware. On a network, logic may be programmed on a server, or a complex of servers. A particular logic unit is not limited to a single logical location on the network. Moreover, the modules need not be executed in any specific order. Each module may call another module when needed to be executed.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or can be included in, one or more separate physical components or media (e.g., multiple CDs, disks, or other storage devices). The operations described in this specification can be implemented as operations performed by a data processing apparatus on data stored on one or more computer-readable storage devices or received from other sources.

Program code embodied on a computer readable medium may be transmitted using any appropriate medium, including but not limited to wireless, wireline, optical fiber cable, R.F, etc., or any suitable combination of the foregoing. Computer program code for carrying out operations for aspects of the present disclosure may be written in any combination of one or more programming languages, including an object oriented programming language such as Java, Smalitalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks.

However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

One or more computing devices such as desktop computers, laptop computers, tablets, smartphones, servers, application-specific computing devices, or any other type(s) of electronic device(s) may be capable of performing the techniques and operations described herein. In some embodiments, the system may be implemented as a single device. In other embodiments, the system may be implemented as a combination of two or more devices together. For example, the system may include one or more server computers and a one or more client computers communicatively coupled to each other via one or more local-area networks and/or wide-area networks such as the Internet.

Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices. To provide for interaction with a user, embodiments of the subject matter described in this specification can be implemented on a computer having a display device, e.g., an LCD (liquid crystal display). LED (light emitting diode) display, or OLED (organic light emitting diode) display, for displaying information to the user. Examples of input devices include a keyboard, cursor control devices (e.g., a mouse or a trackball), a microphone, a scanner, and so forth, wherein the user can provide input to the computer. Other kinds of devices can be used to provide for interaction with a user as well; for example, feedback provided to the user can be in any form of sensory feedback, e.g., visual feedback, auditory feedback, or tactile feedback; and input from the user can be received in any form, including acoustic, speech, or tactile input. Examples of output devices include a display device (e.g., a monitor or projector), speakers, a printer, a network card, and so forth. Display devices may include display devices that provide visual information, this information typically may be logically and/or physically organized as an array of pixels. In addition, a computer can interact with a user by sending documents to and receiving documents from a device that is used by the user; for example, by sending web pages to a web browser on a user's client device in response to requests received from the web browser.

An interface controller may also be included that may comprise any of a variety of known or future software programs for providing input and output interfaces. For example, interfaces may include what are generally referred to as “Graphical User Interfaces” (often referred to as GUI's) that provide one or more graphical representations to a user. Interfaces are typically enabled to accept user inputs using means of selection or input known to those of ordinary skill in the related art. In some implementations, the interface may be a touch screen that can be used to display information and receive input from a user. In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft Windows Powershell that employs object-oriented type programming architectures such as the Microsoft .NET framework.

Those of ordinary skill in the related art will appreciate that interfaces may include one or more GUI's, CLI's or a combination thereof. A processor may include a commercially available processor such as a Celeron, Core, or Pentium processor made by Intel Corporation, a SPARC processor made by Sun Microsystems, an Athlon, Sempron, Phenom, Ryzen or Opteron processor made by AMD Corporation, or it may be one of other processors that are or will become available. Some embodiments of a processor may include a multi-core processor and/or be enabled to employ parallel processing technology in a single or multi-core configuration. For example, a multi-core architecture typically comprises two or more processor “execution cores”. Each execution core may perform as an independent processor that enables parallel execution of multiple threads. In addition, those of ordinary skill in the related will appreciate that a processor may be configured in what is generally referred to as 32 or 64 bit architectures, or other architectural configurations now known or that may be developed in the future.

A processor typically executes an operating system, which may be, for example, a Windows type operating system from the Microsoft Corporation; the Mac OS X op-crating system from Apple Computer Corp.; a Unix or Linux-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks). For example, the network can include one or more local area networks. The computing system can include any number of clients and servers. A client and server are generally remote from each other and typically interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

Also, a computer may include one or more library files, experiment data files, and an internet client stored in system memory. For example, experiment data could include data related to one or more experiments or assays, such as detected signal values, or other values associated with the biomarker expression data. Additionally, an internet client may include an application enabled to access a remote service on another computer using a network and may for instance comprise what are generally referred to as “Web Browsers”, In the present example, some commonly employed web browsers include Microsoft Internet Explorer available from Microsoft Corporation, Nilozilla Firefox from the Mozlla Corporation, Safari from Apple Computer Corp., Google Chrome from the Google Corporation, or other type of web browser currently known in the art or to be developed in the future. Also, in the same or other embodiments an Internet client may include, or could be an element of, specialized software applications enabled to access remote information via a network such as a data processing application for biological applications.

A network may include one or more of the various types of networks known to those of ordinary skill in the art. For example, a network may include a local or wide area network that may employ what is commonly referred to as a TCP/IP protocol suite to communicate. A network may include a network comprising a worldwide system of interconnected computer networks that is commonly referred to as the internet, or could also include various intranet architectures. Those of ordinary skill in the related arts will also appreciate that some users in networked environments may prefer to employ what are generally referred to as “firewalls” (also sometimes referred to as Packet Filters, or Border Protection De-vices) to control information traffic to and from hardware and/or software systems. For example, firewalls may comprise hardware or software elements or some combination thereof and are typically designed to enforce security policies put in place by users, such as for instance network administrators, etc.

When executed, instructions (which may be stored in the memory) cause at least one of the processors of the computer system to receive an input, which is expression data of a panel of metabolic biomarkers in a biological sample obtained from the subject i. Once the necessary inputs are provided, a module is then executed to derive object features and context features and to calculate object feature metrics and context feature metrics. The object feature metrics and context feature metrics are provided to a trained end classifier, which classifies the object and provides an output to the user. The output may be to a display, a memory, or any other means suitable in the art.

### Example

The following are non-limiting examples of the present invention. It is to be understood that said examples are not intended to limit the present invention in any way. Equivalents or substitutes are within the scope of the present invention.

### Example 1: Metabolic Profile of Patients with PAH as Compared to Those with Diabetes Mellitus and Left Heart Disease

The plasma metabolic profiles were examined in patients with idiopathic PAH, patients with type 2 diabetes mellitus (DM) (University of Arizona (UA) Center for Disparities in Diabetes, Obesity, and Metabolism) and patients with left heart diseases (Heart) (UA biobank). Groups were matched for age (mean±STD): 41.1±15.7; 41.8±13.7; 50.2±10.9; and sex female/male (%): 81.8/18.1; 83.3/16.7; 72.7/27.3; for PAH, DM and Heart, respectively.

Targeted metabolomic analysis was used to quantify primary plasma metabolites, including carbohydrates, amino acids, and nucleotides by GC-TOF mass spectrometry. Two runs of profiling were acquired separately, PAH versus DM and PAH versus Heart. In the DM/PAH run, from the 172 metabolites detected, 84 metabolites were significantly altered (p<0.05). A comparison of PAH and Heart groups showed 76 significantly changed metabolites among 158 that were identified. Heat maps in FIG. 1A show characteristic clustering of PAH group, distinguishing them from DM and Heart patients. Principal component analysis (PCA) of significantly changed metabolites showed clear separation of PAH sub-population from DM and Heart patients (FIG. 1B). The results of this analysis indicate that the plasma metabolomics profile is distinctly different in patients with PAH compared to diabetic patients or patients with left heart diseases. Therefore, the plasma metabolome has a strong potential to serve as a diagnostic tool.

Moreover, 32 unique metabolites that are significantly altered in PAH patients compared to either diabetics or left heart patients can be used as a fingerprint for PAH. These unique metabolites can be classified into five major groups.

The primary group of metabolites consists of mitochondrial-derived metabolites, including tricarboxylic acid (TCA) metabolites and their derivatives. Oxalic acid, the product of oxaloacetate decomposition, was increased over 20-fold in PAH. These changes may indicate undergoing anaplerotic reactions resulting in an accumulation of oxaloacetate, as well as other TCA metabolites.

The next largest group of metabolites consists of carbohydrates. This could be an indication of altered glycolysis due to the metabolic shift associated with PAH pathogenesis. Vascular remodeling in PAH could be the main reason for a decreased level of circulating amino acids, the primary building blocks of proteins that are highly consumed by proliferating cells.

The next important group of metabolites that distinguish PAH patients from other patient cohorts related to myo-inositol and its derivatives. Myo-inositol was recently shown to play an important role in proliferative signaling in PAH.

The last two groups of metabolites contain metabolites that are indicative of damage known to be directly associated with PAH, or an altered gut microbiome, which has also been implemented/implicated in PAH pathogenesis.

### Example 2: Metabolic Fingerprint Comprising 11 Metabolites

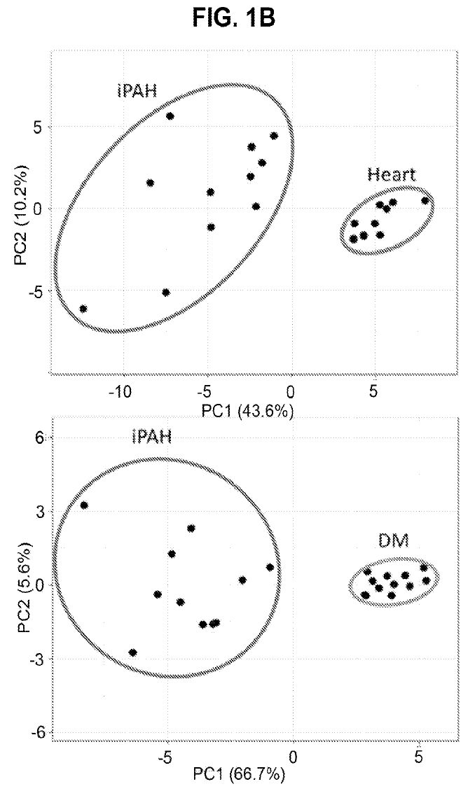

Although the analysis of PAH samples vs. DM or Heart cohorts were assessed in two different runs, a very robust reproducibility of the metabolic data is shown in FIG. 2A, upper panel). The two experiments were then combined using the PAH group as a reference point and comparing all three patients' cohorts together (FIG. 2B, lower panel).

A metabolite optimization approach and regression analysis identified the minimal number of metabolites sufficient for a significant separation of PAH group from DM and Heart. In the lower panel of FIG. 2B, eleven metabolites were used to distinguish PAH samples from other patients. Ellipses showed the area of 0.95 probability that patients from the specific group will fall inside the ellipse area. This analysis indicates that although there is some small intersection between DM and Heart groups, PAH patients were well resolved from both diseases.

The panel of eleven metabolites comprises oxalic acid, pseudouridine, gluconic acid, fumaric acid, UDP-glucuronic acid, aconitic acid, erythritol, 2-deoxytetronic acid, glutamic acid, inorganic phosphate, and 2-hydroxyglutaric acid and can be used for the pre-screening of patients either to identify PAH at the asymptomatic stage or help to minimize the time for PAH diagnosis after the initial symptom onset reported to be currently 47.1±34.2 months.

In summary, the present invention features application or use of metabolic profiling as a new diagnostic method and provides subsequent research with a specific set of preselected metabolites that could serve as a fingerprint of PAH and/or discerning the pathogenesis of PAH.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A computer-implemented method for diagnosing a subject with a disease, the method comprising:
a) inputting into a computer system expression data of a panel of metabolic biomarkers in a biological sample obtained from the subject;
b) determining whether expression of the metabolic biomarkers in the biological sample obtained from the subject is indicative of the disease using the computer system programmed with a trained machine learning classifier for distinguishing subjects with different diseases and without disease;
wherein the machine learning classifier has been trained using expression data of a panel of metabolic biomarkers from subjects having the disease and from control subjects that do not have disease; and
c) diagnosing the subject if the expression data of the panel of metabolic biomarkers in the biological sample obtained from the subject is correlated by the computer system to be indicative of the disease;
where the diagnostic accuracy is at least 90%, and wherein the panel of metabolic biomarkers comprises at least 5 metabolites selected from a group consisting of oxalic acid, aminomalonate, pseudouridine, gluconic acid, isothreonic acid, 4-hydroxyphenylacetic acid, erythritol, uric acid, uridine diphosphate (UDP)-glucuronic acid, fumaric acid, focuse, aconitic acid, 2-deoxytetronic acid, pantothenic acid, indole-3-acetate, myo-inositol, 2-hydroxyvaleric acid, citric acid, ribonic acid, glycine, glutamic acid, creatinine, glucuronic acid, phosphate, indole-3-lactate, urea, 2-hydroxyglutaric acid, tryptophan, tyrosine, glutamine, lysine, histidine, N-acetylornithine, 2-hydroxybutanoic acid, alpha-ketoglutarate, and oxoproline.

2. The method of claim 1, wherein the expression data of the panel of metabolic biomarkers is determined using standard clinical chemistry techniques, protein analytic techniques, nucleic acid techniques, and/or analytical techniques suitable for metabolite analysis.

3. (canceled)

4. The method of claim 1, wherein the trained machine learning classifier is a logistic regression.

5. (canceled)

6. The method of claim 1, wherein the metabolites further consist of adenosine-5-monophosphate, 3-phenyllactic acid, pyrophosphate, maltotriose, glucose-1-phosphate, myristic acid, docosahexaenoic acid, aspartic acid, tocopherol gamma, 5-methoxytryptamine, arachidic acid, cystine, adipic acid, 3-hydroxybutyric acid, cholesterone, 2,4-diaminobutyric acid, and 3-aminoisobutyric acid.

7. The method of claim 1, wherein the metabolites are measured throughout time in samples obtained from the subject longitudinally throughout time, wherein longitudinally throughout time comprises 1) an initial time comprising at time of no symptoms, at time of diagnosis, or at initial presentation of symptoms; 2) about weekly intervals post initial time; 3) about monthly intervals post initial time; and/or 4) about yearly intervals.

8. (canceled)

9. The method of claim 1, wherein the disease comprises pulmonary arterial hypertension (PAH), Diabetes Mellitus (DM), left heart disease, chronic obstructive pulmonary disease (COPD), interstitial lung disease (ILD), head and neck cancer, thyroid cancer or colon cancer.

10.-11. (canceled)

12. The method of claim 1, wherein the method is used for determining the severity of PAH in the patient.

13. The method of claim 1, wherein the method discriminates between pulmonary arterial hypertension (PAH) and classical metabolic disorders, including diabetes mellitus (DM), or left heart diseases.

14.-15. (canceled)

16. The method of claim 1, wherein the biological sample comprises plasma, serum, urine, obtained from the subject.

17. The method of claim 1, wherein the method is used to guide treatment and/or care management of the subject diagnosed with pulmonary arterial hypertension (PAH).

18. A method of treating a subject with pulmonary arterial hypertension (PAH), the method comprising the steps of:
a) determining the expression data of a panel of metabolic biomarkers in a biological sample obtained from the subject;
b) treating PAH in a subject identified as having differing levels of the panel of metabolic biomarkers when compared to the levels of said metabolic biomarkers in a normal subject;
wherein the panel of metabolic biomarkers comprises oxalic acid, aminomalonate, pseudouridine, gluconic acid, erythritol, uric acid, uridine diphosphate (UDP)-glucuronic acid, fumaric acid, aconitic acid, 2-deoxytetronic acid, myo-inositol, citric acid, glutamic acid, phosphate, N-acetylornithine, 2-hydroxybutanoic acid, alpha-ketoglutarate, and oxoproline;
wherein the subject with PAH has a higher level of oxalic acid, aminomalonate, pseudouridine, gluconic acid, uric acid, UDP-glucuronic acid, aconitic acid, 2-deoxytetronic acid, myo-inositol, citric acid, glutamic acid, N-acetylornithine, 2-hydroxybutanoic acid, alpha-ketoglutarate, oxoproline than the normal subject; and
wherein the subject with PAH has a lower level of erythritol, fumaric acid, and phosphate than the normal subject.

19.-20. (canceled)

21. The method of claim 18, wherein the method is used for determining the severity of PAH in the subject.

22.-24. (canceled)

25. The method of claim 18, wherein the treatment of pulmonary hypertension comprises administration of a drug, surgery, transplantation, exercise training, physical rehabilitation, and/or balloon angioplasty.

26. A non-transitory, computer-readable medium having computer executable instructions for causing a processor to execute a method for diagnosing a subject with a disease, the method comprising:
a) determining whether expression data of a panel of metabolic biomarkers in a biological sample obtained from the subject is indicative of the disease using a trained machine learning classifier for distinguishing subjects with different diseases and without disease;
wherein the machine learning classifier has been trained using expression data of a panel of metabolic biomarkers from subjects having the disease and from control subjects that do not have disease; and
b) diagnosing the subject if the expression data is correlated to be indicative of the disease;
where the diagnostic accuracy is at least 90%, and wherein the panel of metabolic biomarkers comprises at least 5 metabolites selected from a group consisting of oxalic acid, aminomalonate, pseudouridine, gluconic acid, isothreonic acid, 4-hydroxyphenylacetic acid, erythritol, uric acid, UDP-glucuronic acid, fumaric acid, focuse, aconitic acid, 2-deoxytetronic acid, pantothenic acid, indole-3-acetate, myo-inositol, 2-hydroxyvaleric acid, citric acid, ribonic acid, glycine, glutamic acid, creatinine, glucuronic acid, phosphate, indole-3-lactate, urea, 2-hydroxyglutaric acid, tryptophan, tyrosine, glutamine, lysine, histidine, N-acetylornithine, 2-hydroxybutanoic acid, al pha-ketoglutarate, and oxoproline.

27. A kit for diagnosing a subject with a disease, the kit comprising:
a) one or more reference metabolic biomarker panels; and
b) a non-transitory, computer-readable medium of claim 26;
wherein expression data of a panel of metabolic biomarkers in a biological sample obtained from the subject is inputted into a computer that executes the computer executable instructions of the non-transitory, computer-readable medium;
wherein the subject is diagnosed with the disease when the expression data of the panel of metabolic biomarkers in the biological sample obtained from the subject is correlated with the one or more reference metabolic biomarker panels by the computer to be indicative of disease; and where the diagnostic accuracy is at least 90%, and wherein the panel of metabolic biomarkers comprises at least 5 metabolites selected from a group consisting of oxalic acid, aminomalonate, pseudouridine, gluconic acid, isothreonic acid, 4-hydroxyphenylacetic acid, erythritol, uric acid, uridine diphosphate (UDP)-glucuronic acid, fumaric acid, focuse, aconitic acid, 2-deoxytetronic acid, pantothenic acid, indole-3-acetate, myo-inositol, 2-hydroxyvaleric acid, citric acid, ribonic acid, glycine, glutamic acid, creatinine, glucuronic acid, phosphate, indole-3-lactate, urea, 2-hydroxyglutaric acid, tryptophan, tyrosine, glutamine, lysine, histidine, N-acetylornithine, 2-hydroxybutanoic acid, alpha-ketoglutarate, and oxoproline.

28. The kit of claim 27, wherein the expression data of the panel of metabolic biomarkers is determined using standard clinical chemistry techniques, protein analytic techniques, nucleic acid techniques, and/or analytical techniques suitable for metabolite analysis.

29. (canceled)

30. The kit of claim 27, wherein the metabolites further consist of adenosine-5-monophosphate, 3-phenyllactic acid, pyrophosphate, maltotriose, glucose-1-phosphate, myristic acid, docosahexaenoic acid, aspartic acid, tocopherol gamma, 5-methoxytryptamine, arachidic acid, cystine, adipic acid, 3-hydroxybutyric acid, cholesterone, 2,4-diaminobutyric acid, and 3-aminoisobutyric acid.

31. The kit of claim 27, wherein the metabolites are measured throughout time in samples obtained from the subject longitudinally throughout time, wherein longitudinally throughout time comprises 1) an initial time comprising at time of no symptoms, at time of diagnosis, or at initial presentation of symptoms; 2) about weekly intervals post initial time; 3) about monthly intervals post initial time; and/or 4) about yearly intervals.

32. (canceled)

33. The kit of claim 27, wherein the disease comprises pulmonary arterial hypertension (PAH), Diabetes Mellitus (DM), left heart disease, chronic obstructive pulmonary disease (COPD), interstitial lung disease (ILD), head and neck cancer, thyroid cancer or colon cancer.

34.-39. (canceled)

40. The kit of claim 27, wherein the biological sample comprises plasma, serum, urine, obtained from the subject.
